(19) United States
(12) Patent Application Publication
(10) Pub. No.: US 2025/0009666 A1
(43) Pub. Date: Jan. 9, 2025

(54) RAPIDLY DISSOLVING SALT TABLET COMPOSITIONS AND METHODS

(71) Applicant: Luisam Tarrats, San Juan, PR (US)

(72) Inventor: Luisam Tarrats, San Juan, PR (US)

(21) Appl. No.: 18/760,369

(22) Filed: Jul. 1, 2024

Related U.S. Application Data
(60) Provisional application No. 63/524,749, filed on Jul. 3, 2023.

(51) Int. Cl. A61K 9/20; A61K 31/351; A61K 31/4045; A61K 31/422; A61K 31/4468; A61K 31/455; A61K 31/485; A61K 31/565; A61K 31/58; A61K 38/09; A61K 38/095; A61K 38/23

(52) U.S. Cl. CPC A61K 9/2072; A61K 9/2009; A61K 9/2013; A61K 9/2031; A61K 9/2095; A61K 31/351; A61K 31/4045; A61K 31/422; A61K 31/4468; A61K 31/455; A61K 31/485; A61K 31/565; A61K 31/58; A61K 38/09; A61K 38/095; A61K 38/23

(57) ABSTRACT

Disclosed are rapidly dissolving salt tablet compositions. Methods for using the rapidly dissolving salt tablets for preparation of isotonic saline solutions for use in nasal irrigation or cleansing are provided.

### CROSS REFERENCE TO RELATED APPLICATIONS

The present application claims the benefit of U.S. Provisional Patent Application Ser. No. 63/524,749, filed on Jul. 3, 2023, which is incorporated herein by reference in its entirety.

### TECHNICAL FIELD

The present disclosure in general is related to salt tablet compositions and methods. More particularly, the present disclosure is related to a rapidly dissolving salt tablet that can be used to prepare a saline solution, for example, that can be used for cleansing or irrigation of the nasal cavity of a user.

### BACKGROUND

Nasal cleansing involves washing out nasal and sinus passages with a suitable solution. The solution is made to circulate in and out of the sinus cavities, coming back out from the mouth or from the opposite side of the user's nose. This is believed to be helpful in maintaining nasal hygiene, limiting sinus infection and in relieving symptoms and signs of rhinitis. Also, in pre and post-operative periods, nasal irrigation can be used to flush out bacteria, clots, and normal crusts.

Nasal irrigation is now widely recognized as a treatment for chronic rhinosinusitis and during the postoperative period. Topical sinus irrigations play a critical role in the management of sinonasal disease, and contributes to the success of endoscopic sinus surgery. The composition of the cleansing or irrigation solution plays an important role in the effectiveness of the treatment. For example the saline concentration can alter the effectiveness of the treatment. Additionally, since the pH of a healthy human nose is about 6.4, solutions that are too high or too low in pH could cause nasal irritation, and lead to reduced compliance with cleansing or irrigation treatment.

Preparation of such cleansing or irrigation solutions for home use can be cumbersome and difficult. Currently salt sachets are used to prepare nasal irrigation or cleansing solutions. However salt sachets present a number of problems. For example many people have problems using nasal irrigation salt sachets when preparing the irrigation solution. In addition salt sachets create a significant amount of paper and plastic waste.

Accordingly, salt tablet compositions and methods that can be used to precisely prepare nasal cleansing or irrigation solutions, and that reduce or eliminate paper and/or plastic waste, are desirable and an object of the present disclosure.

### SUMMARY

The following presents a simplified summary in order to provide a basic understanding of some aspects of the present disclosure. This summary is not an extensive overview, and it is not intended to identify key/critical elements or to delineate the scope thereof. Its sole purpose is to present some concepts in a simplified form as a prelude to the more detailed description that is presented later.

The present disclosure provides a tablet, comprising between about 60% and about 80% by weight of sodium chloride, between about 10% and about 20% by weight of sodium bicarbonate or sodium carbonate, and between about 2% and about 10% by weight of an anhydrous organic acid, wherein the tablet is configured to dissolve. In certain embodiments the tablet comprises about 75% by weight of sodium chloride, about 20% by weight of sodium bicarbonate, and about 5% by weight of an anhydrous organic acid. In some embodiments the anhydrous organic acid is selected from the group consisting of citric acid, carboxylic acid, sulfonic acid, lactic acid, acetic acid, formic acid, trifluoroacetic acid, trichloroacetic acid, succinic acid, salicylic acid, gallic acid, oxalic acid, uric acid, malic acid, methane sulfonic acid, p-toluenesulfonic acid, mandelic acid, picric acid, benzoic acid, glycolic acid, tannic acid, adipic acid, maleic acid, butyric acid, barbituric acid, decanoic acid, azelaic acid, camphoric acid, cyanuric acid, gluconic acid, thiosalicylic acid, valeric acid, cacodylic acid, phthalic acid, thiodiglycolic acid, abietic acid, diglycolic acid, isobutyric acid, cinnamic acid, humic acid, tiglic acid, parnoic acid, glycerophosphoric acid, vanillic acid, tetronic acid, tartronic acid, propionic acid and tartaric acid. In particular embodiments the organic acid is citric acid.

In certain embodiments the tablet is configured to dissolve completely in water in less than about 120 seconds, less than about 90 seconds, less than about 75 seconds, less than about 50 seconds or less than about 40 seconds. In some embodiments the tablet is formed by direct compression. In other embodiments the tablet is round, oval or capsule shaped. In yet other embodiments the tablet has a thickness of between about 20 mm and about 40 mm. In still other embodiments the tablet has a thickness of 35 mm.

In further embodiments the tablet comprises about 2% to about 20% of an excipient. In certain embodiments the excipient is polyethylene glycol, polyvinylpyrrolidone, hydroxymethyl cellulose, hydroxypropyl cellulose or hydroxypropyl methyl cellulose. In some embodiments the excipient is polyethylene glycol. In other embodiments the excipient is polyethylene glycol having a molecular weight of 4,000, 6,000, 8,000 or 10,000. In particular embodiments the excipient is polyethylene glycol having a molecular weight of 10,000.

In still further embodiments the tablet comprises a drug. In certain embodiments the drug is a small molecule or a peptide or protein. In some embodiments the drug is a small molecule. In various embodiments the drug is zolmitriptan, sumatriptan, butorphanol tartrate, fentanyl, nicotine polacrilex, 17beta-estradiol. budesonide, mometasone, mupirocin, a steroid or an antibiotic. In other embodiments the drug is a peptide or protein. In particular embodiments the drug is calcitonin, desmopressin, buserelin, nafarelin, or oxytocin. In yet further embodiments the tablet comprises an absorption promoter, enhancer or modulator. In certain embodiments the absorption promoter, enhancer or modulator is cyclopentadecalactone, sodium N-[8-(2-hydroxybenzoyl)amino] caprylate, 8-(N-2-hydroxy-5-chloro-benzoyl)-amino-caprylic acid, sodium caprate, alkylsaccharides, chitosan, low methylated pectin, or polyglycol mono- or diesters of 12 hydroxystearate.

### BRIEF DESCRIPTION OF THE DRAWINGS

All of the figures depict preferred embodiments, although other embodiments are contemplated, and the present disclosure is not limited to the embodiments shown.

FIG. 1 shows the tablet masses for five different acidic tablet formulations.

FIG. 2 shows the tablet masses for five different basic tablet formulations.

FIG. 3 shows the tablet widths for five different acidic tablet formulations.

FIG. 4 shows the tablet widths for five different basic tablet formulations.

FIG. 5 shows the measured sodium chloride concentration for five different acidic tablet formulations when dissolved in 300 ml of double deionized water.

FIG. 6 shows the pH value of an acidic tablet formulation when dissolved in three different types of water.

FIG. 7 shows the pH value of a basic tablet formulation when dissolved in three different types of water.

FIG. 8 shows the change in pH value over time of an acidic tablet formulation and a basic tablet formulation.

FIG. 9 shows the tensile strength of five different acidic tablet formulations.

FIG. 10 shows a comparison of tensile strength versus compression depth for two different compression depths (three tablets per study).

FIG. 11 shows the time required to dissolve for five different acidic tablet formulations and five different basic tablet formulations.

FIG. 12 shows the results shown in FIG. 11 with a focus on the lower times.

FIG. 13 shows an example of a tableting/encapsulation process flow chart.

### DETAILED DESCRIPTION

To provide an overall understanding of the disclosure, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and those described herein may be employed in other suitable applications, and such other additions and modifications will not depart from the scope hereof.

In the following detailed description, numerous specific details are set forth in order to provide a thorough understanding of the present disclosure. However, it will be understood by those skilled in the art that the present disclosure may be practiced without these specific details. In other instances, well-known methods, procedures and components have not been described in detail so as not to obscure the present disclosure.

The present disclosure provides a fast dissolving salt tablet that can be used to prepare an isotonic saline solution for nasal irrigation or cleansing, and additionally in some embodiments for the delivery of one or more drugs to the nasal passages. The primary ingredients in nasal irrigation solutions are sodium chloride and sodium bicarbonate or sodium carbonate. In order to make a salt tablet that rapidly dissolves, the rate of solubility of the tablet should be increased. The rate of solubility for any substance is defined by the Noyes-Whitney equation:

$$\frac{dC}{dT} = {kS ( {C}_{S} - {C}_{t} )}_{\cdot }$$

where k is the dissolution rate constant, S is the surface area, Cₛ is the theoretical saturation concentration, and Cₜ is the current concentration. From the equation above, the dissolution rate (k) and theoretical saturation concentration (Cₛ) are fixed (constant) physical properties that cannot be changed or influenced. Heat can increase the Cₛ thereby improving the rate of solubility; however, the effects are limiting. The surface area, that is the amount of water exposed to the salts, can be altered, and the concentration of these salts (Cₜ) can also be changed. Given that the target concentration of sodium chloride and sodium bicarbonate (or sodium carbonate) is generally fixed in an isotonic saline nasal rinse (0.9 wt % and 0.3 wt %, respectively), Cₜ is a fixed value and cannot be changed. Therefore, the only parameter that can be changed to improve the rate of solubility (dC/dT) is the surface area (S).

There are two methods to increase the surface area between the salts and the water. First, stirring the mixture will improve the contact between the water and salt tablet allowing for faster dissolution. Second, formulating a porous tablet will increase surface area significantly, because water will be able to interact with regions inside the salt tablet that could not be exposed with a compact, non-porous design. To create pores within the tablet structure immediately after contact with water, the tablet is prepared by compounding an anhydrous organic acid with bicarbonate, which results in a violent reaction when placed in water because both species react to form carbon dioxide gas. The consumption of the organic acid and bicarbonate in the solid tablet forms pores that increase the surface are between the water and other salts, which significantly improves dissolution through an increase in surface area. The formation of carbon dioxide bubbles helps to agitate the tablet and further improve mixing.

The inventors have created a rapidly dissolving salt tablet that can be used to prepare an isotonic saline cleansing or irrigation solution by resolving four separate issues. First, the choice of organic acid, since different organic acids will have different impacts on tablet solubility and stability. Second, the concentration of organic acid and bicarbonate, since optimal balance between both ingredients should be achieved for dissolution without affecting pH of saline cleansing or irrigation solution. Third, the tablet shape and size, since the tablet shape and size should be optimized for rapid solubility. And fourth, optimization of compounding and mixture preparation, since manufacturing at scale requires thorough mixing of all ingredients with special considerations to prevent settling of ingredients, thereby creating tablets with varied ingredient concentrations.

In various embodiments the volume of the isotonic saline solution can vary, depending on the particular application or use. For example the volume of saline solution can be about 1 ml, about 5 ml, about 10 ml, about 25 ml, about 50 ml, about 75 ml, about 100 ml, about 150 ml, about 200 ml, about 250 ml, about 300 ml, about 400 ml, about 500 ml, about 600 ml, about 700 ml, about 800 ml, about 900 ml, about 1000 ml, about 1250 ml, about 1500 ml, about 1750 ml or about 2000 ml or more, or any amount in between the foregoing amounts. Although the volume of the isotonic saline solution can vary, the ratio of the components of the solution remains essentially constant.

The tablet according to certain embodiments comprises between about 60% and about 80% by weight of sodium chloride, between about 10% and about 20% by weight of sodium bicarbonate or sodium carbonate, and between about 2% and about 10% by weight of an anhydrous organic acid, wherein the tablet is configured to dissolve. For example, the sodium chloride may comprise about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80% by weight, or any percentage in between the foregoing percentages. For example, the sodium bicarbonate or sodium carbonate may comprise about 10%, about 11%, about 12%, about 13%, about 14%, about 15%, about 16%, about 17%, about 18%, about 19%, or about 20% by weight, or any percentage in between the foregoing percentages. For example, the anhydrous organic acid may comprise about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, and about 10% by weight, or any percentage in between the foregoing percentages.

In certain embodiments the tablet comprises about 75% by weight of sodium chloride, about 20% by weight of sodium bicarbonate, and about 5% by weight of an anhydrous organic acid.

In some embodiments the anhydrous organic acid is selected from the group consisting of citric acid, carboxylic acid, sulfonic acid, lactic acid, acetic acid, formic acid, trifluoroacetic acid, trichloroacetic acid, succinic acid, salicylic acid, gallic acid, oxalic acid, uric acid, malic acid, methane sulfonic acid, p-toluenesulfonic acid, mandelic acid, picric acid, benzoic acid, glycolic acid, tannic acid, adipic acid, maleic acid, butyric acid, barbituric acid, decanoic acid, azelaic acid, camphoric acid, cyanuric acid, gluconic acid, thiosalicylic acid, valeric acid, cacodylic acid, phthalic acid, thiodiglycolic acid, abietic acid, diglycolic acid, isobutyric acid, cinnamic acid, humic acid, tiglic acid, parnoic acid, glycerophosphoric acid, vanillic acid, tetronic acid, tartronic acid, propionic acid and tartaric acid, or any combinations of the foregoing. In particular embodiments the organic acid is citric acid.

In certain embodiments the tablet is configured to dissolve completely in water in less than about 120 seconds, less than about 90 seconds, less than about 75 seconds, less than about 50 seconds or less than about 40 seconds. For example, the tablet may dissolve completely in water in about 120 seconds, about 115 seconds, about 110 seconds, about 105 seconds, about 100 seconds, about 95 seconds, about 90 seconds, about 85 seconds, about 80 seconds, about 75 seconds, about 70 seconds, about 65 seconds, about 60 seconds, about 59 seconds, about 58 seconds, about 57 second, about 55 seconds, about 54 seconds, about 53 seconds, about 52 seconds, about 51 seconds, about 50 seconds, about 49 seconds, about 48 seconds, about 47 seconds, about 46 seconds, about 45 seconds, about 44 seconds, about 43 seconds, about 42 seconds, about 41 seconds, about 40 seconds, about 39 seconds, about 38 seconds, about 37 seconds, about 36 seconds, about 35 seconds, about 34 seconds, about 33 seconds, about 32 seconds, about 31 seconds, or about 30 seconds, or any amount of time in between the foregoing.

In some embodiments the tablet is formed by direct compression. In other embodiments the tablet is round, oval, ovoid, polygonal, or capsule shaped.

In yet other embodiments the tablet has a thickness of between about 20 mm and about 40 mm. In still other embodiments the tablet has a thickness of 35 mm. For example, the tablet may have a thickness of about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, or about 40 mm, or any thickness in between the foregoing.

In further embodiments the tablet comprises about 2% to about 20% of an excipient. For example, the tablet may comprise an excipient in an amount of about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, or any percentage in between the foregoing.

In certain embodiments the excipient is polyethylene glycol, polyvinylpyrrolidone, hydroxymethyl cellulose, hydroxypropyl cellulose or hydroxypropyl methyl cellulose, or any combinations of the foregoing. In some embodiments the excipient is polyethylene glycol. In other embodiments the excipient is polyethylene glycol having a molecular weight of about 4,000, about 4,100, about 4,200, about 4,300, about 4,400, about 4,500, about 4,600, about 4,700, about 4,800, about 4,900, about 5,000, about 5,100, about 5,200, about 5,300, about 5,400, about 5,500, about 5,600, about 5,700, about 5,800, about 5,900, about 6,000, about 6,100, about 6,200, about 6,300, about 6,400, about 6,500, about 6,600, about 6,700, about 6,800, about 6,900, about 7,000, about 7,100, about 7,200, about 7,300, about 7,400, about 7,500, about 7,600, about 7,700, about 7,800, about 7,900, about 8,000, 8,100, about 8,200, about 8,300, about 8,400, about 8,500, about 8,600, about 8,700, about 8,800, about 8,900, about 9,000, about 9,100, about 9,200, about 9,300, about 9,400, about 9,500, about 9,600, about 9,700, about 9,800, about 9,900, about 10,000, or any molecular weight in between any of the foregoing. In particular embodiments the excipient is polyethylene glycol having a molecular weight of 10,000.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Chemical Composition of Salt Tablets

Various formulations of salt tablets were studied to determine the effect on the characteristics of the tablets. Different ratios of sodium chloride, sodium bicarbonate, polyethylene glycol (PEG) and citric acid were used to produce tablets. Tablets were comprised of pharmaceutical grade sodium chloride (CAS #7440-23-5) and sodium bicarbonate (CAS #144-55-8), and in some cases 10K PEG (CAS #25322-68-3) and citric acid (CAS #77-92-9). The active ingredient is sodium chloride, and was used in an amount to prepare 300 ml of isotonic saline for a sinus rinse (0.9%), Sodium bicarbonate and citric acid are used to maintain the pH and to enhance dissolution by increasing surface area, and PEG is used to improve tablet strength and uniformity. Tablets containing citric acid were referred to as “acidic” tablets, and tablets without citric acid were referred to as “basic” tablets. The acidic tablets were compounded with citric acid to improve solubility kinetics and to achieve a final pH of ˜6.5. Five different acidic tablet dry salt formulations are shown in Table 1, below, and five different basic tablet dry salt formulations are shown in Table 2, below.

TABLE 1
Acidic Tablet Formulation by Weight % in Dry Salt Mixture

| Tablet | Final | % Sodium | % | % Sodium | % Citric |
|---|---|---|---|---|---|
| # | pH | Chloride | PEG | Bicarbonate | Acid |
| 1 | ~6.5 | 75 | 0 | 20 | 5 |
| 2 | ~6.5 | 71.25 | 5 | 19 | 4.75 |
| 3 | ~6.5 | 67.5 | 10 | 18 | 4.5 |
| 4 | ~6.5 | 60 | 20 | 16 | 4 |
| 5 | ~6.5 | 52.5 | 30 | 14 | 3.5 |

TABLE 2
Acidic Tablet Formulation by Weight % in Dry Salt Mixture

| Tablet | Final | % Sodium | % Sodium | % |
|---|---|---|---|---|
| # | pH | Chloride | Bicarbonate | PEG |
| 1 | ~7.6 | 99 | 1 | 0 |
| 2 | ~7.6 | 94.05 | 0.95 | 5 |
| 3 | ~7.6 | 89.1 | 0.9 | 10 |
| 4 | ~7.6 | 79.2 | 0.8 | 20 |
| 5 | ~7.6 | 69.3 | 0.7 | 30 |

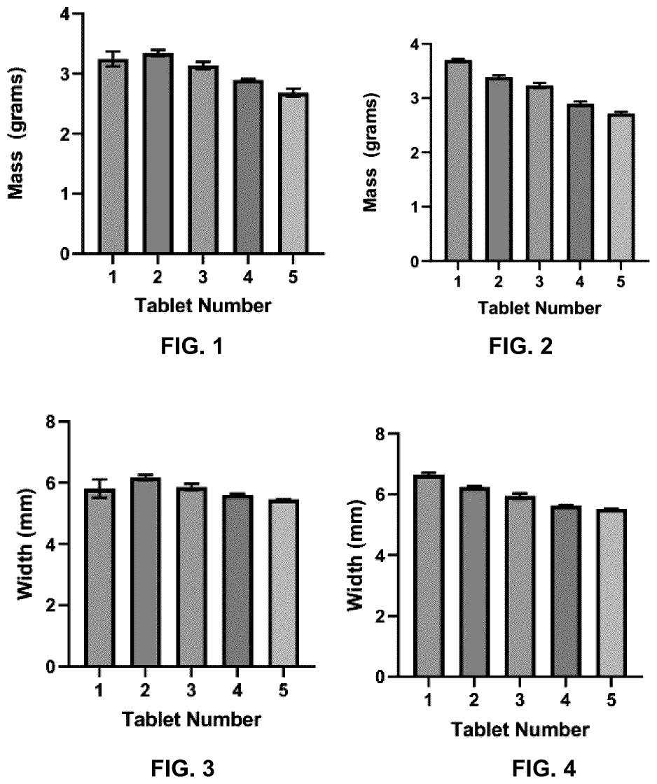

The tablet masses for the acidic tablet formulations is shown in FIG. 1, the tablet masses for the basic tablet formulations is shown in FIG. 2, tablet widths for the acidic tablet formulations is shown in FIG. 3, and tablet widths for the basic tablet formulations is shown in FIG. 4. The results show that each formulation has a different tablet weight and thickness, even though the same manufacturing procedure was used for all of the tablets. The pill weights and thicknesses show the addition of PEG-10k decreases the total mass of the pill in addition to the total volume. This is likely because PEG is significantly less dense than the salts used in the saline tablet. The decreased total volume and pill mass is not significant from 0% to 30% PEG (less than a 25% decrease in pill weight and volume), showing that the 0.9% saline concentration in 300 ml of water using PEG as an excipient can be achieved.

The weight percent of sodium chloride was determined for the acidic tablets and the five basic tablets. The results for the acidic tablets are shown in Table 3, below, and the results for the basic tablets are shown in Table 4, below.

TABLE 3
Acidic Tablet NaCl wt % in 300 ml H₂O

| | | | Weight |
|---|---|---|---|
| | Average | % | % NaCl |
| Tablet | Mass | NaCl in | in 300 ml |
| # | (g) | Tablet | H₂O |
| 1 | 3.70 | 75 | 0.93 |
| 2 | 3.39 | 71.25 | 0.80 |
| 3 | 3.23 | 67.5 | 0.73 |
| 4 | 2.90 | 60 | 0.58 |
| 5 | 2.72 | 52.5 | 0.48 |

TABLE 4
Basic Tablet NaCl wt % in 300 ml H₂O

| | | | Weight |
|---|---|---|---|
| | Average | % | % NaCl |
| Tablet | Mass | NaCl in | in 300 ml |
| # | (g) | Tablet | H₂O |
| 1 | 3.24 | 99 | 1.07 |
| 2 | 3.34 | 94.05 | 1.05 |
| 3 | 3.13 | 89.1 | 0.93 |
| 4 | 2.90 | 79.2 | 0.76 |
| 5 | 2.68 | 69.3 | 0.62 |

Since the sodium chloride weight percent in isotonic saline is 0.9%, the results confirm that the desired weight percent of sodium chloride can be attained using a single tablet.

Next three different tablet manufacturing techniques were studied. One kilogram powder mixtures (pre-ground salts with particle sizes of less than 5 μm) were prepared by weighing each component into a 2 L drum to the nearest 5 mg (each component added as a % of 1 kg as outlined in the prior study). The following mixing procedures were used: 1) Blending with overhead stirrer—salt mixture was blended with an overhead stirrer with 10 cm paddles for 60 minutes; 2) Drum mixing—salt mixture was blended in salt drum for 30 minutes (rotational mixing only); and 3) Speed mixing—the salt mixture was blended in a speed mixer with 1 mm stainless steel beads for 5 minutes prior to compounding. The mixed salts were then compounded on a Natoli pill press using a 20 mm casting die, 35 mm die casting thickness, and 15 kN of force. The pill press was pre-lubricated with magnesium stearate. The recovered pills were then packaged and stored under ambient conditions until used.

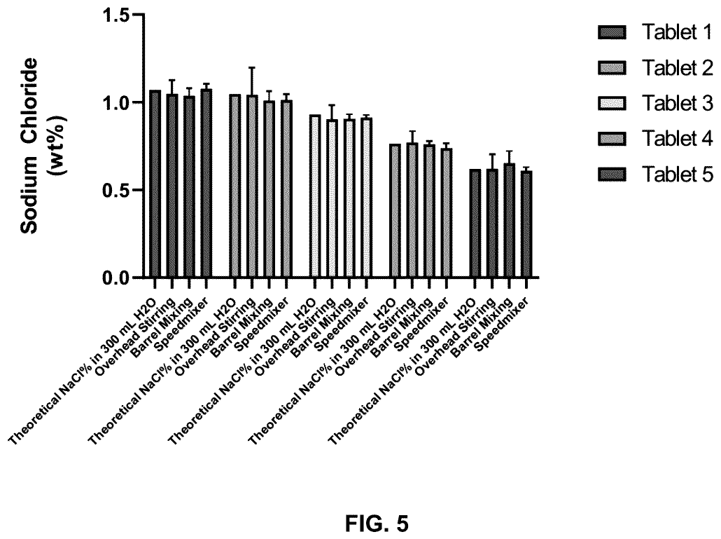

The results are shown in FIG. 5, which shows the sodium chloride concentration from 1 tablet in 300 ml of double deionized (DDI) water measured by ion chromatography. All data sets show the mixing methods are very close to the theoretical values of NaCl wt %. The data shows that overhead stirring is the least consistent method for the blending of the salt mixture given the large uncertainties in sodium chloride concentration, likely because there are spots in the chamber where the stirring blades cannot agitate successfully. Drum mixing and speed-mixing appear to be very close in performance with deviations of less than 5%. Pills compounded at a sub 15 mm thickness were brittle and were significantly weaker than pills compounded at 35 mm. Thicker pills had a better compression edge and were significantly more robust than thinner tablets. Pre-blending/grinding each of the powders to a sub 5 micron particle size improved the strength of the tablet significantly.

### Example 2—Tablet pH

The saline tablet was tuned through the preparation of the salt mixture to optimize various tablet characteristics, including: 1) saline concentration; 2) mechanical strength; 3) pH of final saline rinse solution.; and 4) solubility kinetics. Optimization of the pH of the final saline solution was achieved through the careful incorporation of sodium bicarbonate and citric acid (when used in the tablet) to achieve specific buffered solutions.

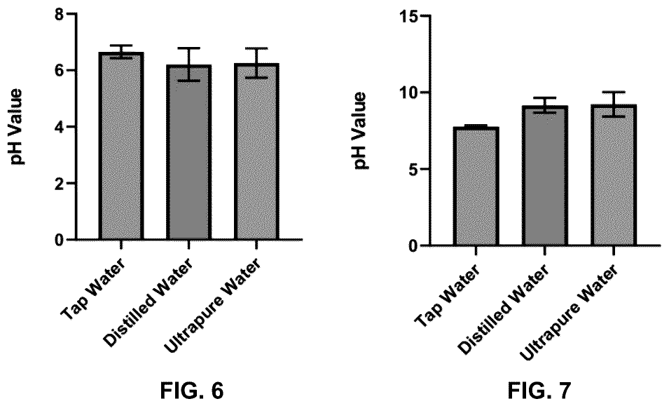

Acidic tablets were formulated to achieve a pH between 6.0-6.5. Basic tablets were formulated to achieve a pH between 7.5-8.0. Both types of tablets were formulated with 10% PEG-10k. The pH of the acidic and basic tablets were tested upon dissolution in three different water types, tap water, distilled water, and ultrapure water. The results for the acidic tablets are shown in FIG. 6, and the results for the basic tablets are shown in FIG. 7. The results show that the acidic tablets can maintain the pH in all water types, whereas the basic tablets are influenced by the metals/ions, or lack thereof, in the water.

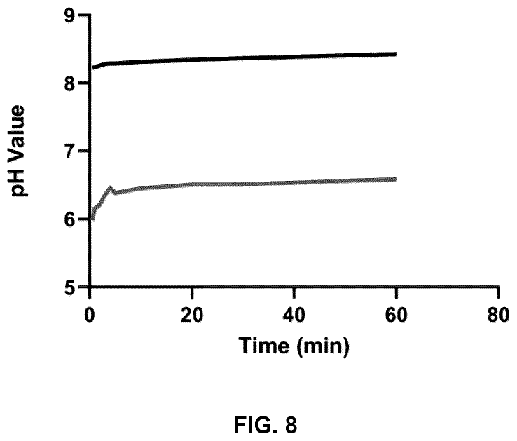

Since a bicarbonate buffer is being used, CO₂ gas is evolved from the dissolution of bicarbonate from the solution thereby changing the solution pH. A kinetics study was conducted to measure the change in pH over the course of 60 minutes, using both the acidic and basic tablets with 10% PEG dissolved in deionized water. The results are shown in FIG. 8. The results show the pH of the acidic and basic solutions remain stable over time in deionized water.

### Example 3—Mechanical Strength

Studies were conducted to increase the mechanical strength of the saline tablet. As shown above, thin tablets were extremely fragile and required careful handling to prevent edge damage and/or disintegration of the tablet. To improve the mechanical strength of the tablet, two factors were studied. First, PEG-10k was added. PEG is known to be an excellent excipient in tablet compounding to increase the strength of pills. Second, the tablet depth/size was optimized. An Instron (Norwood, MA) universal testing machine (UTM) was used to measure the tensile (snapping) strength of the tablet. A metal die was pushed on the center of the die until the tablet snapped. The resulting force curve was used to compare the strength of each formulation.

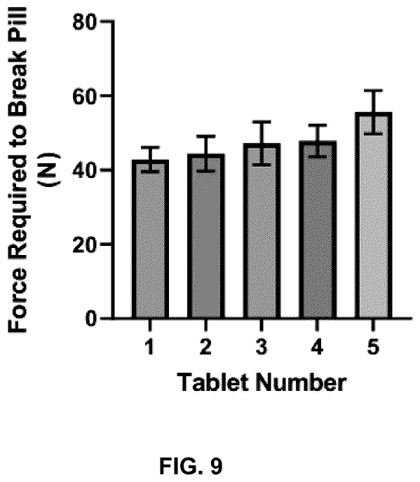

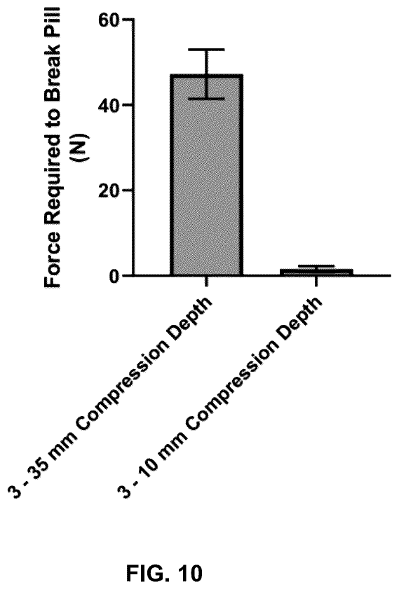

The results of the tensile strength measurements obtained by the addition of PEG are shown in FIG. 9. The results show the incorporation of PEG did improve the tensile strength of the pills; however, the improvement was minimal (less than 14% unless 30% of PEG-10k is added). The results of studies on the effect on tensile strength using various thicknesses of the tablet are shown in FIG. 10. Compression depth (tablet thickness) had a significant improvement on tablet tensile strength. The studies showed that thicker tablets with a die setting >20 mm significantly improved the tablet strength and robustness.

The results showed that the addition of a tableting excipient, such as PEG, only has mild improvement on tablet tensile strength except at the highest concentration of PEG. Formulations containing 30% PEG had a significant improvement in tensile strength due to having a critical compaction concentration of PEG needed to achieve tighter packing. Formulations with 0 to 20% PEG only show minor differences in strength. Compression depth had a significant impact on tensile strength. Increasing the compression depth by a factor of 3.5 improved the tensile strength by a factor of 25-40×. All formulations, except for formulations containing 30% PEG-10k, were susceptible to edge damage/chipping, although the edge chipping/damage was less as the percentage of PEG increased.

### Example 4—Solubility

Solubility of the various acidic and basic tablet designs were studied and evaluated in 300 mL of deionized water. The purpose of this study was to identify a set of conditions that favors rapid dissolution of a salt tablet to prepare 300 ml of saline solution at pH 6.5.

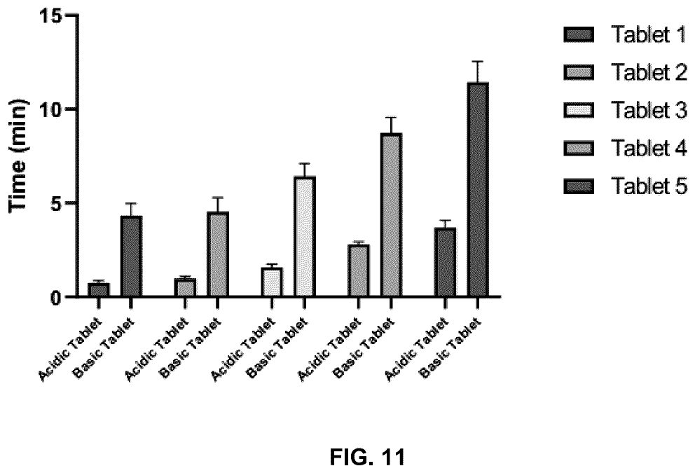

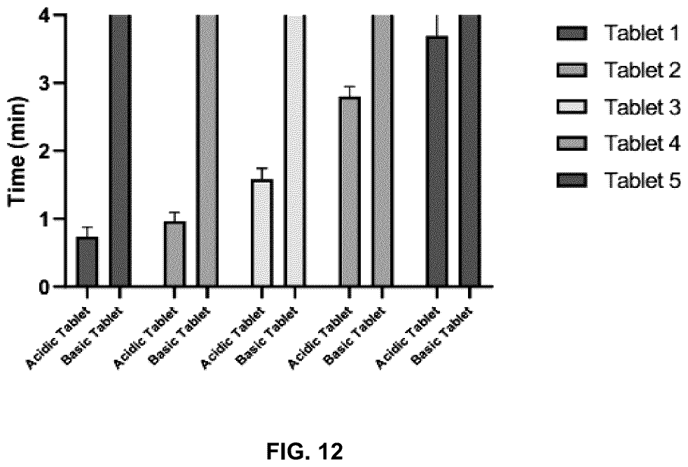

The results of the studies to determine the time required to dissolve various formulations of the saline tablets are shown in FIG. 11. FIG. 12 shows the same results as shown in FIG. 11 but focusing on the shorter times. The results show that the incorporation of citric acid is important for hastening the solubility of the saline tablets, due to an increase in surface area between the salt and water. The tablets without citric acid and tablets containing greater than 10% PEG-10k took much longer (more than 2 minutes) to fully dissolve, likely due to tighter packing of the salt tablet and poorer solubility when compared with native salts. Other water-soluble excipients such as polyvinylpyrrollidone (PVP) and hydroxymethyl cellulose (HMC) improved tablet strength, but had poorer solubility than PEG-10k. PEG-5k and PEG-100k had similar effects on solubility. However, in formulations containing non-water soluble drugs, the addition of excipients, such as PEG or PVP, can be used as surfactants to help solubilize non-water soluble drugs in aqueous solution for nasal delivery. In such formulations the slower dissolution kinetics are a valuable trade-off for better solubilization of non-water soluble drugs.

### Example 5—Tableting/Encapsulation Process Flow

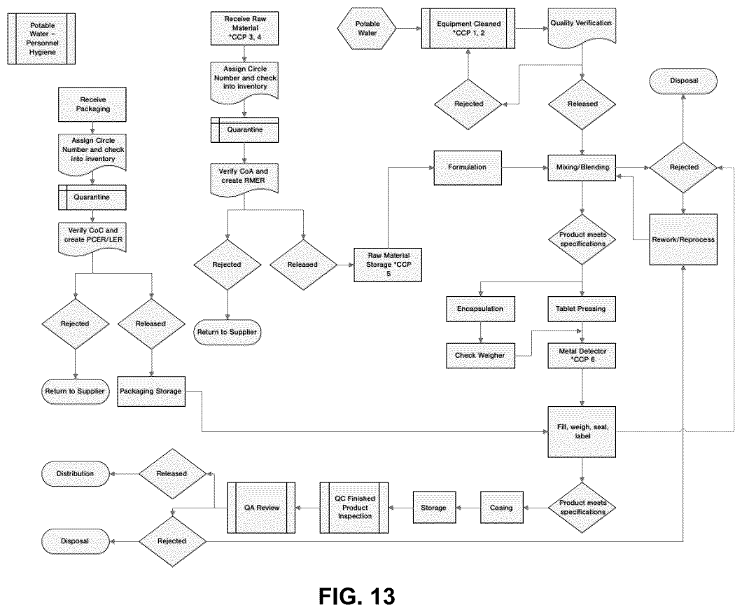

An example of a flow chart of the tableting/encapsulation process is shown in FIG. 13. A description of an example of the process is shown below.

1) Materials for final product are tested for proper identification before manufacturing occurs. 2) Using an identified partial screen, each material is mixed in a blender (standard) mixing unit, and the material is screened though an 80 mesh screen to verify and create uniform partials for the Salt, PEG-8000, PVP-K-90, citric acid, and sodium bicarbonate. 3) After the materials are charged in the blender they are mixed and sampled at 5, 10, and 20 minute intervals. 4) Room temperature is held between 60-70° F., and relative humidity (rH) 40-45% rH. This is a unique method allowing lower rH in product production where formulation is based on functional break down of PEG-8000/PVP-K-90 blend. Concentration of citric acid and sodium bicarbonate are too low for full effervescent statement. 5) Product is bulked off, sealed and sampled. This is held until process uniformity testing is completed for bulk material. 6) Material then is transferred to tablet press area (room temperature is 55-65° F., and rH is held between 35-50% rH). 7) Press set up. a) Range of tablet setting is 8-10 ton. Running double rotary style press with tooling. b) Coating of tooling at this point is 17% Chrome based tooling, no extra release coating on tooling is used, 440C stainless steel is this composition of the tooling and die-set. Tooling is 22 mm round with minor beveled edges. 8) Product held in bulk is pressed and held for testing uniformity (average kiloponds (Kp) 7-10 range for final tablet hardness, dissolving rate of tablet using purified water at 90° F. is 15-30 seconds or less, linearity of weight range is 5% of 3.25 g final mass of tablet, product is held before packaging—finished packaging in single dose sashes' or desiccant containers (for larger counts)).

Consideration of unique properties of formation. 1) PEG-8000 as a wax property, combined with PVP-K-90 form a based super disintegrating base, also for deliverable for forming salt tablet increasing tinsel strength of finished tablet without reducing speed of dissolving. 2) PEG's and PVP style super disintegrants create a value to reduce direct breakdown in dry format of sodium bicarbonate, and citric acid, reducing stability profile of the two active pharmaceutical ingredients (APIs) defined in the final product stated as sodium chloride and sodium bicarbonate. 3) Additives as oils, glidants (microcrystalline cellulose—MCC) as insoluble compounds or releasing agents (calcium stearate, steric acid, magnesium stearates or other fat based systems) which are insoluble compounds that are common in drug finished product are not needed for reducing time and systems which is fully soluble as components obtained in this finished formulation noted above. 4) Additives noted above reduce dissolving rate of tablets in general use and practices for processing and manufacturing of tablet, and are known as each has hydrophobic properties to each additive. 5) With addition of PEG's and PVP (hydrophilic) polymers dissolve in water efficiently, but do not have an effect on pH structure of the final solution or have effect on osmotic change of the components of sodium chloride, sodium bicarbonate and citric acid (note given average range of diluted solution with tablet added to purified water, or distilled water; PEG and PVP has no direct effect on final pH of suitable base of composition).

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Features or functionality described with respect to certain example embodiments may be combined and sub-combined in and/or with various other example embodiments. Also, different features and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

As used herein, a term “about” or “substantially” refers to a +/−10% variation from a nominal value/term. Such variation is always included in any given value/term provided herein, whether or not such variation is specifically referred thereto.

Any component described herein can include a material suitable for a medical use. The material can be, flexible, elastic, or resilient. The material can be suitable to be disinfected, sterilized, or sanitized, which can be with a hot steam, an autoclave, or others. For example, the material can include plastic, metal, rubber, shape memory, fabric, foam, or others.

The device and system of the present disclosure has been described with specific reference to certain drawings and various embodiments, but may, however, be embodied in many different forms and should not be construed as necessarily being limited to only embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected” or “coupled” to another element, then the element can be directly on, connected or coupled to the other element or intervening elements can be present, including indirect or direct variants. In contrast, when an element is referred to as being “directly connected” or “directly coupled” to another element, there are no intervening elements present.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

As used in the specification and claims, the singular forms “a”, “an” and “the” include plural references unless the context clearly dictates otherwise. For example, the term “material” may include a plurality of materials unless the context clearly dictates otherwise. As used in the specification and claims, singular names or types referenced include variations within the family of said name unless the context clearly dictates otherwise. For example, a term “a” or “an” shall mean “one or more,” even though a phrase “one or more” is also used herein.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the various parts of the nasal irrigation device or any assembly of them may be used.

Moreover, terms “comprises,” “includes” or “comprising,” “including” when used in this specification, specify a presence of stated features, integers, steps, operations, elements, or components, but do not preclude a presence and/or addition of one or more other features, integers, steps, operations, elements, components, or groups thereof. Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

Additionally, although terms first, second, and others can be used herein to describe various elements, components, regions, layers, or sections, these elements, components, regions, layers, or sections should not necessarily be limited by such terms. Rather, these terms are used to distinguish one element, component, region, layer, or section from another element, component, region, layer, or section. As such, a first element, component, region, layer, or section discussed herein could be termed a second element, component, region, layer, or section without departing from this disclosure.

Also, unless otherwise defined, all terms (including technical and scientific terms) used herein have the same meaning as commonly understood by one of ordinary skill in an art to which this disclosure belongs. As such, terms, such as those defined in commonly used dictionaries, should be interpreted as having a meaning that is consistent with their meaning in a context of a relevant art and should not be interpreted in an idealized or overly formal sense unless expressly so defined herein.

In addition, features described with respect to certain example embodiments may be combined in or with various other example embodiments in any permutational or combinatory manner. Different features or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Although preferred embodiments have been depicted and described in detail herein, skilled artisans know that various modifications, additions, substitutions and the like can be made without departing from spirit of this disclosure. As such, these are considered to be within the scope of the disclosure, as defined in the following claims.

1. A tablet, comprising between about 60% and about 80% by weight of sodium chloride, between about 10% and about 20% by weight of sodium bicarbonate or sodium carbonate, and between about 2% and about 10% by weight of an anhydrous organic acid, wherein the tablet is configured to dissolve.

2. The tablet of claim 1, wherein the tablet comprises about 75% by weight of sodium chloride, about 20% by weight of sodium bicarbonate, and about 5% by weight of an anhydrous organic acid.

3. The tablet of claim 1, wherein the anhydrous organic acid is selected from the group consisting of citric acid, carboxylic acid, sulfonic acid, lactic acid, acetic acid, formic acid, trifluoroacetic acid, trichloroacetic acid, succinic acid, salicylic acid, gallic acid, oxalic acid, uric acid, malic acid, methane sulfonic acid, p-toluenesulfonic acid, mandelic acid, picric acid, benzoic acid, glycolic acid, tannic acid, adipic acid, maleic acid, butyric acid, barbituric acid, decanoic acid, azelaic acid, camphoric acid, cyanuric acid, gluconic acid, thiosalicylic acid, valeric acid, cacodylic acid, phthalic acid, thiodiglycolic acid, abietic acid diglycolic acid, isobutyric acid, cinnamic acid, humic acid, tiglic acid, parnoic acid, glycerophosphoric acid, vanillic acid, tetronic acid, tartronic acid, propionic acid and tartaric acid.

4. The tablet of claim 3, wherein the organic acid is citric acid.

5. The tablet of claim 1, wherein the tablet is configured to dissolve completely in water in less than about 120 seconds, less than about 90 seconds, less than about 75 seconds, less than about 50 seconds or less than about 40 seconds.

6. The tablet of claim 1, wherein the tablet is formed by direct compression.

7. The tablet of claim 1, wherein the tablet is round, oval or capsule shaped.

8. The tablet of claim 1, wherein the tablet has a thickness of between about 20 mm and about 40 mm.

9. The tablet of claim 8, wherein the tablet has a thickness of 35 mm.

10. The tablet of claim 1, further comprising about 2% to about 20% of an excipient.

11. The tablet of claim 10, wherein the excipient is polyethylene glycol, polyvinylpyrrolidone, hydroxymethyl cellulose, hydroxypropyl cellulose or hydroxypropyl methyl cellulose.

12. The tablet of claim 11, wherein the excipient is polyethylene glycol.

13. The tablet of claim 12, wherein the excipient is polyethylene glycol having a molecular weight of 4,000, 6,000, 8,000 or 10,000.

14. The tablet of claim 13, wherein the excipient is polyethylene glycol having a molecular weight of 10,000.

15. The tablet of claim 1, further comprising a drug.

16. The tablet of claim 15, wherein the drug is a small molecule or a peptide or protein.

17. The tablet of claim 16 wherein the drug is a small molecule.

18. The tablet of claim 17 wherein the drug is zolmitriptan, sumatriptan, butorphanol tartrate, fentanyl, nicotine polacrilex, 17beta-estradiol. budesonide, mometasone, mupirocin, a steroid or an antibiotic.

19. The tablet of claim 16 wherein the drug is a peptide or protein.

20. The tablet of claim 19 wherein the drug is calcitonin, desmopressin, buserelin, nafarelin, or oxytocin.

21. The tablet of claim 1, further comprising an absorption promoter, enhancer or modulator.

22. The fast dissolving tablet of claim 21, wherein the absorption promoter, enhancer or modulator is cyclopentadecalactone, sodium N-[8-(2-hydroxybenzoyl)amino] caprylate, 8-(N-2-hydroxy-5-chloro-benzoyl)-amino-caprylic acid, sodium caprate, alkylsaccharides, chitosan, low methylated pectin, or polyglycol mono- or diesters of 12-hydroxystearate.
